(19) United States
(12) Patent Application Publication
(10) Pub. No.: US 2004/0157920 A1
(43) Pub. Date: Aug. 12, 2004

(54) Method of adenovirus, HIV and HPV prophylaxis

(76) Inventor: Claudia Cherney Stewart, Southampton, NY (US)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/662,848

(22) Filed: Sep. 15, 2003

Related U.S. Application Data
(63) Continuation of application No. 09/611,286, filed on Jul. 6, 2000, now abandoned.
Application No. 09/611,286 is a continuation-in-part of application No. 09/330,629, filed on Jun. 11, 1999.
(60) Provisional application No. 60/142,480, filed on Jul. 6, 1999.
(60) Provisional application No. 60/142,577, filed on Jul. 7, 1999.

(51) Int. Cl. A61K 31/28

(52) U.S. Cl. 514/492

(57) ABSTRACT

The likelihood of infection by a broad spectrum of viruses, including Adenovirus, Human Immunodeficiency Virus and/or Human Papillomavirus infection can be reduced by the prophylactic topical application, prior to the exposure to or infection by the virus of metallo-organic cobalt compounds according to the following formula to the site of infection:

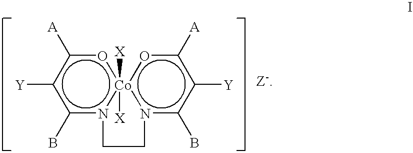

wherein each A may be the same or different and is an alkyl group, a phenyl group or a substituted derivative of a phenyl group;

wherein each Y may be the same or different and is hydrogen, an unbranched alkyl group, a halide or a group having the structure

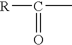

wherein R is hydrogen, an alkoxide group, and alkyl group, or OH;

wherein each B may be the same or different and each is hydrogen or an alkyl group;

wherein each X may be the same or different and each is a water soluble group having weak to intermediate ligand field strength; and

Z⁻ is a soluble, pharmaceutically acceptable negative ion.

Metallo-organic cobalt compounds may also be used to disinfect liquids which contain adenovirus, Human Immunodeficiency Virus and/or Human Papillomavirus.

### CROSS REFERENCE TO RELATED APPLICATIONS

This application is a continuation-in-part application of U.S. patent application Ser. No. 09/330,629, filed on Jun. 11, 1999. This application claims the benefit of priority of U.S. Patent Application Nos. 60/142,480, filed on Jul. 6, 1999, and 60/142,577, filed on Jul. 7, 1999.

### BACKGROUND OF THE INVENTION

The present invention relates to metallo-organic cobalt compounds and their use in the prophylactic treatment of subjects to prevent or reduce the risk of transmission of viruses such as adenovirus (Ad), human immunodeficiency virus (HIV) and/or human papillomavirus (HPV).

It has been discovered that certain conditions and diseases, e.g., inflammation, burns, wounds, and diseases caused by bacteria and fungi in mammalian species can be treated with certain complexes of cobalt having the structure:

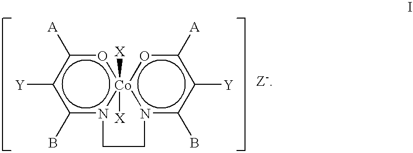

wherein each A may be the same or different and is an alkyl group, a phenyl group or a substituted derivative of a phenyl group;

wherein each Y may be the same or different and is hydrogen, an unbranched alkyl group, a halide or a group having the structure

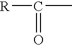

wherein R is hydrogen, an alkoxide group, and alkyl group, or OH;

wherein each B may be the same or different and each is hydrogen or an alkyl group;

wherein each X may be the same or different and each is a water soluble group having weak to intermediate ligand filed strength; and

Z⁻ is a soluble, pharmaceutically acceptable negative ion.

U.S. Pat. No. 5,142,076, discloses the use of the foregoing described compounds as treatment for viral diseases.

U.S. patent application Ser. No. 09/306,165, filing date of May 6, 1999, and Ser. No. 09/330,629, filing date of Jun. 11, 1999, discloses the use of the foregoing described compounds-as prophylactic treatments for herpes viruses, human papillomavirus and human immunodeficiency virus. These applications are hereby incorporated by reference.

Today, virus infections are known to be significant causes of morbidity and mortality in human and veterinary medicine. Many of these diseases are untreatable or the available therapies are not entirely satisfactory and only provide minimal clinical response. New prophylactic treatments would decrease the incidence of these diseases and improve overall health.

### SUMMARY OF THE INVENTION

I have discovered a prophylactic use for the series of compounds having the structure:

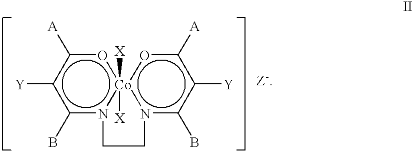

herein

each A may be the same or different and is an alkyl group, a phenyl group or a substituted derivative of a phenyl group;

each Y may be the same or different and is hydrogen, an unbranched alkyl group, a halide or a group having the structure

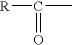

wherein R is hydrogen, an alkoxide group, an alkyl group, or OH;

each B may be the same or different and each is hydrogen or an alkyl group;

Z⁻ is a soluble, pharmaceutically acceptable negative ion; and

each X may be the same or different and is an axial ligand selected from the group consisting of moieties having the formula:

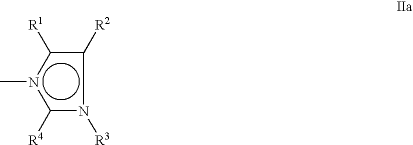

wherein R¹, R², R³, and R⁴ may be the same or different and maybe hydrogen or lower alkyl having from 1 to 4 carbon atoms; and

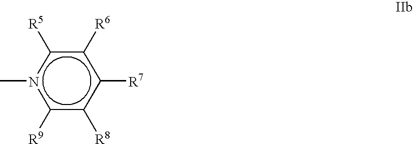

wherein R⁵, R⁶, R⁷, R⁸ and R⁹ may be the same or different and may be selected from the group consisting of electron donating groups and electron withdrawing groups,

with the proviso that R¹, R², R³, R⁴, R⁵, R⁶, R⁷, R⁸, and R⁹, are of a sufficiently small size so as not to prohibit the attachment of the axial ligand to the Co atom due to stearic hindrance.

As used herein, the term “axial” when used in conjunction with the term “ligand” refers to the fact that the ligand is oriented outside the plane of the molecule and has the same meaning as described in connection with FIG. 1 of U.S. Pat. No. 5,049,557. As used herein, and unless otherwise indicated, an alkyl group means a linear, branched or cyclic alkyl group containing from one to six carbon atoms.

The compounds having the structure of Formula II exhibit prophylactic efficacy when applied as a topical composition to the contact site prior to contact with a virus such as adenovirus, HIV or HPV, and/or by inactivating the virus exposed to the composition, and/or by preventing expression of the viral disease. The compositions of the invention may further be used for antisepsis or disinfection of surfaces, such as surgical tools, or preparations, such as media or blood-derived products.

The compounds having the structure of Formula II may be applied for prophylaxis of diseases caused by a broad spectrum of unrelated viruses including but not limited to, adenovirus, herpesvirus, human papillomavirus and human immunodeficiency virus essentially as described in U.S. patent application Ser. No. 09/306,165, filing date of May 6, 1999, and Ser. No. 09/330,629, filing date of Jun. 11, 1999.

### BRIEF DESCRIPTION OF THE DRAWINGS

FIG. 1 is a scatterplot of the effect of prophylactic treatment of Human Papillomavirus on the size of infected skin grafts in an animal model.

### DETAILED DESCRIPTION OF THE INVENTION

The compounds used in the present invention may be crystallized with numerous counter-anions. Counter-anions which are pharmaceutically acceptable and are water soluble, such as, halide ions, PF₆⁻ and BF₄⁻, are preferred. The bromide and chloride salts of the present compounds are the most preferred because they are more water soluble than other salts of the compounds.

As discussed above, A may be an alkyl group, a phenyl group or a substituted derivative of a phenyl group. Preferably, the alkyl group is a C₁-C₅ group with methyl, ethyl, and butyl groups being particularly preferred. Suitable substituted derivatives of the phenyl group are derivatives wherein each substituent is a halide, an alkyl group or a group having the structure

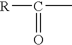

where R is hydrogen, an alkoxide group, an alkyl group or an OH group. To date, the most useful derivatives have proven to be those in which the substituents are halides, or alkyl groups.

Y may be hydrogen, an unbranched alkyl group, a halide or a group having the structure

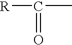

wherein R is hydrogen, an alkoxide group, an alkyl group, or an OH group. In certain embodiments, it is preferred that Y is chlorine, hydrogen atom or a C₁-C₃ alkyl group. In embodiments where Y has a structure

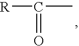

it is preferred that R is hydrogen, a methyl group, or an OH group.

B may be hydrogen or an alkyl group, and preferably is a C₁-C₃ alkyl group.

X may be imidazole or pyridinyl groups linked to the cobalt atom through a nitrogen of the ring. The imidazole or pyridinyl nuclei may have hydrogen atoms, or electron donating or withdrawing groups substituted thereon.

The electron withdrawing or donating groups which may constitute appendant groups R¹, R², R³, R⁴, R⁵, R⁶, R⁷ and R⁸ are those known in the art to exert the specified electron withdrawing or donating effects on aromatic nuclei. Typical of electron donating groups are NO₂⁻, Cl⁻, Br⁻, and the like. The identity of the particular group is not crucial so long as it does not impart properties to the molecule which are detrimental to the desired properties of the compound, e.g., decreased antiviral activity, increased toxicity, and the like. Additionally, the group must not be so large as to prevent the axial ligand to attach to the cobalt atom due to steric effects, e.g., steric hindrance.

Preferably, the groups attached to the imidazole nucleus are alkyl having from one to three carbon atoms. Of these, methyl and ethyl are most preferred. Preferred are the unsubstituted, 2-methyl, 4-methyl, and 2-ethyl imidazoles and the unsubstituted pyridinyl.

The following Table provides the structures of preferred compounds in accordance with the present invention. Compound 23, disclosed in the U.S. Pat. No. 5,142,076 as exhibiting antiviral activity, is included as a comparison in the examples that follow.

In the following diagram, B is, in each case, methyl, and A, Y, X and Z⁻ refer to those symbols as used in structure II.

| COMPOUND | Y | X | Z | A |
|---|---|---|---|---|
| 23 | H | —NH₃ | Cl | —CH₃ |
| 76 | H | 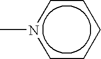 | Br | —CH₃ |
| 82 | H | 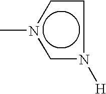 | Cl | CH₃ |
| 93 | Cl | 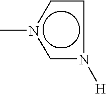 | Br | —CH₃ |
| 96 | H | 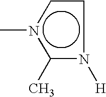 | Br | —CH₃ |
| 97 | H | 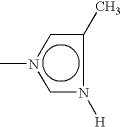 | Br | —CH₃ |
| 98 | H | 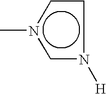 | Br | C₆H₅ |
| 100 | Cl | 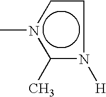 | Br | —CH₃ |
| 101 | Cl | 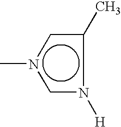 | Br | —CH₃ |
| 102 | H | 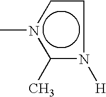 | Cl | C₆H₅ |
| 109 | H | 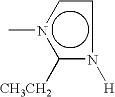 | Cl | —CH₃ |

The compositions used in the instant invention comprise a pharmaceutically acceptable carrier and a compound as defined above in an adenovirus prophylactic effective amount. As used herein, the expressions adenovirus prophylactic effective amount, dosage or regimen mean that amount, dosage or regimen which results in a sufficient concentration of the particular compound at an appropriate site to prevent adenovirus disease. By appropriate site, it is meant a site which potentially contains adenovirus or is an area of a subject of potential exposure to adenovirus or is an area of a subject that has been exposed to adenovirus but as a result of such exposure, the subject has not yet acquired adenovirus disease. As used herein, the expression acquired adenovirus disease means that the subject, in fact, has the disease and can no longer be treated prophylactically to prevent symptoms of the disease and must be treated therapeutically to ameliorate the disease.

The compounds and compositions may be used in preventing infections caused by a variety of adenovirus types, including, but not limited to Ad1, Ad2, Ad3, Ad4, Ad5, Ad6, Ad7, Ad8, Ad11, Ad14, Ad19, Ad21, Ad34, Ad35, Ad37, Ad40 or Ad41. Disease states caused by these viruses include acute febrile pharyngitis, pharyngoconjunctival fever, acute respiratory disease, pneumonia, epidemic keratoconjunctivitis, pertussis-like syndrome, acute hemorrhagic cystitis, gastroenteritis or hepatitis. Certain compounds within the group may exhibit greater efficacy against specified types as compared with other compounds within the inventive group. Accordingly, the present invention includes the inventive compositions wherein the composition contains a compound as defined hereinabove in a prophylactic amount which is effective against the specific adenovirus type.

The compositions used in the instant invention also comprise a pharmaceutically acceptable carrier and a compound as defined above in an HIV and/or HPV prophylactic effective amount. As used herein, the expressions HIV and/or HPV prophylactic effective amount, dosage or regimen means that amount, dosage or regimen which results in a sufficient concentration of the particular compound at an appropriate site to prevent HIV and/or HPV disease. By appropriate site, it is meant a site which potentially contains HIV and/or HPV or is an area of a subject of potential exposure to HIV and/or HPV disease or is an area of a subject that has been exposed to HIV and/or HPV disease but as a result of such exposure, the subject has not yet acquired HIV or HPV disease. As used herein, the expression acquired HIV or HPV disease means that the subject, in fact, has the disease and can no longer be treated prophylactically to prevent symptoms of the disease and must be treated therapeutically to ameliorate the disease.

The compounds and compositions may also be used in preventing infections caused by a variety of HIV or HPV types, such as HIV-1, HIV-2, HPV-1, HPV-2, HPV-3, HPV-4, HPV-6, HPV-7, HPV-10, HPV-11, HPV-16, HPV-18, HPV-31 or HPV-45. Certain compounds within the group may exhibit greater efficacy against specified types as compared with other compounds within the inventive group. Accordingly, the present invention includes the inventive compositions wherein the composition contains a compound as defined hereinabove in a prophylactic amount which is effective against the specific HIV or HPV type.

The compositions used in the instant invention also comprise a pharmaceutically acceptable carrier and a compound as defined above in a virus prophylactic effective amount. As used herein, the expression virus prophylactic effective amount, dosage or regimen means that amount, dosage or regimen which results in a sufficient concentration of the particular compound at an appropriate site to prevent a viral disease. By appropriate site, it is meant a site which potentially contains a virus or is an area of a subject of potential exposure to a virus or is an area of a subject that has been exposed to a virus but as a result of such exposure, the subject has not yet acquired the virus. As used herein, the expression acquired the virus means that the subject, in fact, has the disease and can no longer be treated prophylactically to prevent symptoms of the disease and must be treated therapeutically to ameliorate the disease.

Known viruses of clinical significance are disclosed in PDR Medical Dictionary, 1st Edition, Williams & Wilkins, pp. 1939-1947, (1995); Virology, B. N. Fields, D. M. Knipe, P. M. Howley, R. M. Chanock, J. L. Melnick, T. P. Monath, B. Roizman and S. E. Straus, Lippincott-Raven Press, N.Y. (1996). See also Antiviral Agents and Viral Diseases of Man, George J. Galasso, Richard J. Whitley, and Thomas C. Merigan, Ed., 4^{th} Edition, Lippincott-Raven Press, N.Y. (1997).

The compounds are particularly effective against, inter alia, HIV-1, HIV-2, HPV-1, HPV-2, HPV-3, HPV-4, HPV-6, HPV-7, HPV-10, HPV-11, HPV-16, HPV-18, HPV-31 and HPV-45.

For topical administration, the inventive composition may be placed in a pharmaceutically acceptable saline solution, ointment, salve, cream or the like. The compounds used in the present invention are water soluble, although the degree of solubility may vary from compound to compound, and may be dissolved in a number of conventional pharmaceutically acceptable carriers. Suitable carriers include polar, protic solvents, such as, water, or normal saline. The compounds may also be suspended in a suspension medium that is not miscible with water, for example, petrolatum.

When the compounds of formula II are to be administered by the topical route for prevention of infection, i.e., prophylaxis or disinfection, their concentration in the saline, ointment, salve, creme, or the like can vary from about 0.00005 to about 5% by weight. A preferred concentration range lies between about 0.0005 and about 2% by weight.

Preferably, the topical concentration is applied to a mucosal lining of the subject, such as in the subject's respiratory tract or eyes. Particularly, preferable is the application to the lungs of the subject in the form of an aerosol spray.

With respect to prophylactic treatment of viruses, a topical composition typically shows prophylactic effect when applied to the contact site from about 8 hours before contact with the virus to about 6 hours after contact with the virus. Preferably, the topical composition is applied from about 1 hour before contact with the virus to about 6 hours after contact.

With respect to prophylactic treatment of adenovirus, a topical composition typically shows prophylactic effect when applied to the contact site from about 8 hours before contact with the virus to about 6 hours after contact with adenovirus. Preferably, the topical composition is applied from about 1 hour before contact with the virus to about 6 hours after contact.

With respect to prophylactic treatment of HIV or HPV, a topical composition typically shows prophylactic effect when applied to the contact site from about 1 hour before contact with the virus to about 6 hours after contact with HIV or HPV. Preferably, the topical composition is applied within five minutes of contact with HIV or HPV. Particularly, the inventive compositions can be applied intravaginally for the prevention of sexual transmission of HIV and/or HPV. The topical composition containing the inventive compound could, for example, be coated on a condom or other sexual barrier device.

When the compounds of formula II are to be used for disinfecting liquid preparations, such as, media, blood-derived products or the like, their concentration in the liquid preparations is from about 0.00005 to about 5% by weight. A preferred concentration range lies between about 0.005% and about 2% by weight. A most preferred concentration range lies between about 0.01% and about 2% by weight.

General methods for the synthesis of the compounds of the present invention are described in U.S. Pat. No. 5,049,557, referred to and incorporated by reference hereinabove. As noted therein, the reaction of Co(II) complexes with molar oxygen has been studied extensively (see, R. S. Drago and B. R. Corden, Acc. Chem. Res., 1980, 13, 353 & E. C. Niederhoffer, J. H. Timmons and A. E. Martell, Chem. Rev. 1984, 84, 137). Normally, cobalt (II) forms 2:1 peroxo bridged complexes in aqueous solutions (see E. C. Niederhoffer, J. H. Timmons and A. E. Martell, Chem. Rev. 1984, 84, 137). In recent years, a number of Co(II) complexes have been reported to give 1:1 cobalt-oxygen adducts at room temperature. These complexes usually contain ligands which when bound to Co(II) give rise to a low spin planar geometry. Addition of base and O₂ to these complexes leads to the formation of octahedral complexes where the base and the O₂ occupy axial positions (see, A. Summerville, R. D. Jones, B. M. Hoffman and F. Basolo, J. Chem. Educ., 1979, 56, 3, 157).

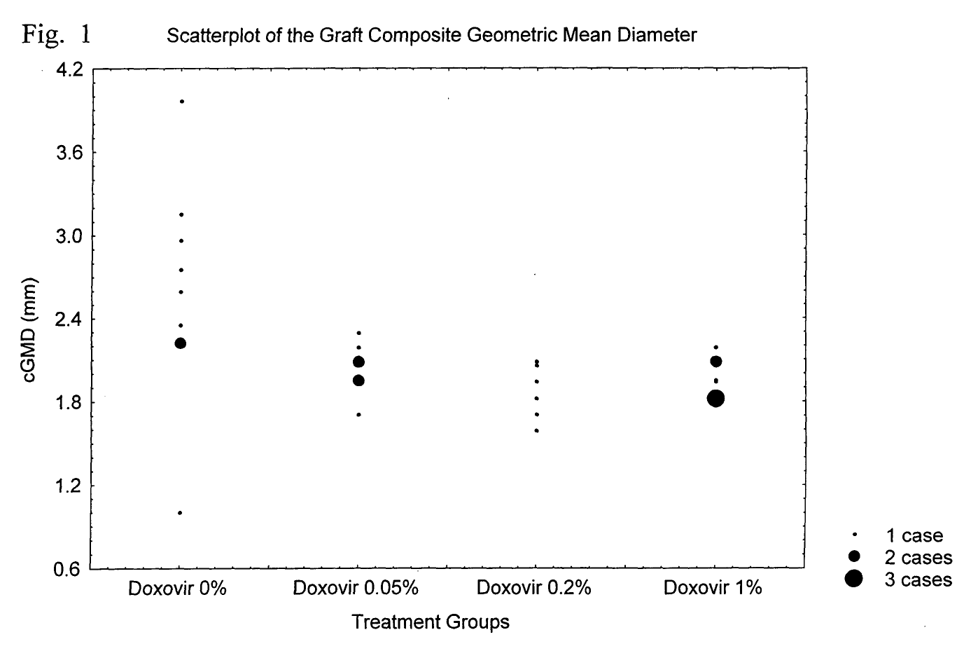

On the basis of measurements utilizing a variety of physical techniques, it is now a well-accepted fact that the most accurate electronic structure description of the Co:O₂ moiety is a Co(III) ion bound to O₂⁻, where the actual amount of Co→O₂ electron transfer depends on the nature of the ligand and the donor set (see, A. Summerville, R. D. Jones, B. M. Hoffman and F. Basolo, J. Chem. Educ. 1979, 56, 3 157, & D. Getz, E. Malmud, B. L. Silver and Z. Dori, J. Am Chem. Soc., 1975, 97, 3846). It has been shown that electron transfer increases with increase of the ligand field strength (see, R. S. Drago and B. R. Corden, Acc. Chem. Res., 1980, 13, 353). This can be easily understood from the molecular orbital diagram depicted in FIG. 1 of U.S. Pat. No. 5,049,557 and the description therein.

The following examples illustrate the present invention. The methods used in the examples are described in the following references:

For in vivo activity and toxicity of antiviral drugs, see Antiviral Agents and Viral Diseases of Man, supra. In particular, Chapter 3, Preclinical Evaluation of Antiviral Agents; In vitro and Animal Model Testing by Dr. Earl R. Kern; Chapter 6, Major Ocular Viral Infections by Dr. Deborah Pavan-Langston; and Chapter 10, Respiratory Diseases.

### EXAMPLE 1

### Preparation of Prophylactic Compounds

The compounds of the present invention may be prepared by the following general procedure. The cobalt-II complex is prepared by mixing equimolar amounts of the N,N′-bisethylenediimine ligands, e.g., L23 and the like as disclosed in U.S. Pat. No. 5,049,557 with cobalt acetate in methanol under nitrogen. About 2.2 equivalents of the desired axial ligand is added followed by oxidation. The desired product may then be precipitated by the addition of a saturated aqueous solution of sodium chloride or sodium bromide followed by recrystallization from an ethanol-water solution.

Compound 96 (having bromide as the counterion) was synthesized as follows:

A 3-neck flask equipped with a nitrogen bubbler and a 2 liter dropping funnel was charged with 112 grams (0.5 moles) of the ligand (L23 or N,N′bis-(acetylacetone)ethylene-diimine) in 500 ml of absolute methanol. To the ligand solution is added 125 grams (0.5 moles) of cobalt acetate tetrahydrate dissolved in 1.5 liters of degassed methanol. The reaction mixture is stirred for 2 hours and then refluxed for 15 minutes on a hot water bath. An orange solution results to which 90 grams (1.1 moles) of 2-methyl imidazole dissolved in 100 ml of methanol are added. The reaction mixture is exposed to the open air while maintaining vigorous stirring. Ten grams of activated charcoal are added to the stirring mixture and the oxidation is continued overnight.

The mixture is then filtered and 50 grams of sodium bromide dissolved in a minimum amount of water is added to the filtered brown solution. The solution obtained is concentrated and allowed to crystallize. The crude product is recrystallized from hot ethanol-water solution by standing at room temperature or a lower temperature. The purity of the product is checked by elemental analysis, electronic spectra and NMR.

### EXAMPLE 2

Compound 96 inhibited adenovirus infection 100% in 293T cells in vitro.

### EXAMPLE 3

Compound 96 inhibited adenovirus infection 67% in A549 cells in vitro.

### EXAMPLE 4

Compound 96 inhibited varicella-zoster virus infection >98% in vitro.

### EXAMPLE 5

### In Vitro Assays with HIV

In a study of the prophylactic effects of Compound 96 on Human Immunodeficiency Virus (HIV), virus stock solutions were inactivated by treatment with Compound 96. Stock solutions of viral types NL-HX-ADA having a p24 concentration of 4.1 μg/ml and NL-HX (P122) having a p24 concentration of 3.8 μg/ml were used for the study, along with a stock solution of Compound 96 having a concentration of 20 mg/ml in RPMI media.

Concentrated stock solution of viruses were treated for 1 hr with equal volume of medium containing the drug at the following concentrations:

TABLE 1
Prophylactic Treatments

| | | | [Cmpd 96] during | |
|---|---|---|---|---|
| | | | incubation | |
| Initial [Cmpd 96] | | | with virus | |
| A. | 20 | mg/ml | 10 | mg/ml |
| B. | 10 | mg/ml | 5 | mg/ml |
| C. | 5 | mg/ml | 2.5 | mg/ml |
| D. | 2.5 | mg/ml | 1.25 | mg/ml |
| E. | 1.25 | mg/ml | 0.6125 | mg/ml |

| | | | |
|---|---|---|---|
| F. | control - | 0 | mg/ml |
| | no drug | | |

After the 1 hr treatment period, the virus-drug solutions were diluted 1:1,000 with complete medium, and then mixed with an equal volume of Peripheral Blood Mononucleocyte (PBMC) cells, for a final dilution of virus and drug of 1:4,000. This resulted in a final virus concentration of 1 ng/ml p24, and the following final drug concentrations:

TABLE 2
Residual concentration of Compound 96
present during incubation with cells

| A. | 5 μg/ml |
|---|---|
| B. | 2.5 μg/ml |
| C. | 1.25 μg/ml |
| D. | 0.6 μg/ml |
| E. | 0.3 μg/ml |
| F. | 0 |

The infected cultures were then incubated without any washing or other changes in media for 4 days (for the M-tropic NL-HX-ADA type) and 8 days (for the T-tropic NL-HX type). The cultures were then assayed for infection using standard fluorescent focus assays to quantitate infected cells.

Control infections were performed by adding untreated viruses at a p24 concentration of 1 ng/ml into cultures containing the final dilution of the drug. This controlled for any residual effect the diluted drug may have on the health of the PBMC culture and their ability to replicate virus.

For both the T cell-tropic (NL-HX) and macrophage-tropic (NL-HX-ADA) virus, the treated viruses were completely inactivated, even at the lowest concentration of drug tested (0.625 mg/ml). No toxicity or inhibition was seen for the control cultures receiving the diluted drugs, even at the highest concentration tested (5 μg/ml).

Below, the actual numbers of infected cells counted per unit area are reported. No infected cells were detected for any of the drug-treated samples, even when the entire well was scanned, whereas control infection had 800-1200 infected cells per well. This indicates at least a 1,000-fold reduction in infectivity. In fact, this data supports the conclusion that a complete inactivation of virus was achieved.

TABLE 3
Treatment of NL-HX-ADA (macrophage-tropic isolate) Virus

| | Drug conc. (mg/ml): | | | | | |
|---|---|---|---|---|---|---|
| | no drug | 10 | 5 | 2.5 | 1.25 | 0.625 |
| # infected cells/ | 14 | 0 | 0 | 0 | 0 | 0 |
| area (˜500 cells) | 15 | 0 | 0 | 0 | 0 | 0 |
| | 15 | 0 | 0 | 0 | 0 | 0 |
| | 15 | 0 | 0 | 0 | 0 | 0 |
| | 16 | 0 | 0 | 0 | 0 | 0 |
| | 16 | 0 | 0 | 0 | 0 | 0 |
| | 17 | 0 | 0 | 0 | 0 | 0 |
| | 17 | 0 | 0 | 0 | 0 | 0 |
| | 17 | 0 | 0 | 0 | 0 | 0 |
| | 16 | 0 | 0 | 0 | 0 | 0 |
| Average # | 15.8 | 0 | 0 | 0 | 0 | 0 |
| infected cells/area | | | | | | |
| Control culture: | 14 | 15 | 16 | 17 | 16 | 17 |
| | 17 | 16 | 17 | 18 | 15 | 16 |
| | 16 | 17 | 15 | 17 | 16 | 16 |
| | 17 | 16 | 15 | 15 | 17 | 17 |
| | 16 | 15 | 17 | 17 | 17 | 18 |
| | 15 | 17 | 16 | 16 | 16 | 16 |
| | 17 | 16 | 16 | 16 | 16 | 16 |
| | 16 | 16 | 18 | 16 | 18 | 16 |
| | 16 | | | | | |
| | 16 | | | | | |
| Average # | 16.00 | 16.00 | 16.25 | 16.50 | 16.38 | 16.50 |
| infected cells/area | | | | | | |

TABLE 4
Virus: NL-HX (T cell-tropic isolate) Virus

| | Drug conc. (mg/ml): | | | | | |
|---|---|---|---|---|---|---|
| | no drug | 10 | 5 | 2.5 | 1.25 | 0.625 |
| # infected cells/ | 11 | 0 | 0 | 0 | 0 | 0 |
| area (˜500 cells) | 10 | 0 | 0 | 0 | 0 | 0 |
| | 10 | 0 | 0 | 0 | 0 | 0 |
| | 9 | 0 | 0 | 0 | 0 | 0 |
| | 11 | 0 | 0 | 0 | 0 | 0 |
| | 10 | 0 | 0 | 0 | 0 | 0 |
| | 10 | 0 | 0 | 0 | 0 | 0 |
| | 11 | 0 | 0 | 0 | 0 | 0 |
| | 11 | 0 | 0 | 0 | 0 | 0 |
| | 11 | 0 | 0 | 0 | 0 | 0 |
| Average # | 10.4 | 0 | 0 | 0 | 0 | 0 |
| infected cells/area | | | | | | |
| Control: | 10 | 10 | 10 | 10 | 9 | 11 |
| culture | 11 | 10 | 10 | 10 | 8 | 10 |
| | 8 | 9 | 8 | 11 | 11 | 10 |
| | 10 | 10 | 8 | 10 | 10 | 10 |
| | 10 | 11 | 9 | | | |
| | 8 | 9 | 8 | | | |
| | 9 | 9 | 8 | | | |
| | 9 | 10 | 9 | | | |
| Average # infected | 9.4 | 9.8 | 8.8 | 10.3 | 9.5 | 10.3 |
| cells/area | | | | | | |

### EXAMPLE 6

### Titration of Anti-HIV Effect of CTC 96

General Methodology:

Stock virus: NL-HX-ADA˜5.5 μg/ml p24 (This is a molecularly cloned macrophage-tropic type of HIV-1). Virus was used to infect cells at 1:4,000 final dilution (˜1.4 ng/ml p24).

Stock CTC-96:10 mg/ml prepared directly in RPMI 1640

Method: Concentrated stock solution of virus was treated for 1 hour with equal volume of medium containing the drug at the indicated concentrations (1:2 dilution). The virus/drug mixture was then diluted 1,000-fold (1:2,000 dilution) and then added to an equal volume of PHA-activated humans PBMCs (1:4,000 final dilution) to allow virus replication to occur. The extent of infection was measured after 4 days by a fluorescent focus assay and compared to that of a control culture not containing any drug. No residual toxicity of CTC-96 was detected under these conditions (highest concentration=2.5 ng/ml).

TABLE 5
Experiment 1:
Extended dose responses for inactivation of HIV-1 by CTC96

| | Initial | [CTC96] during | [CTC96] after | |
|---|---|---|---|---|
| | [CTC96] | incubation with | final dilution | % |
| Batch | mg/ml | virus mg/ml | ng/ml | Neutralization |
| A | 10 | 5 | 2.5 | 100 |
| B | 5 | 2.5 | 1.25 | 100 |
| C | 2.5 | 1.25 | 0.61 | 100 |
| D | 1.25 | 0.625 | 0.31 | 100 |
| E | 0.6 | 0.312 | 0.156 | 83 |
| F | 0.3 | 0.156 | 0.078 | 63 |
| G | 0.156 | 0.078 | 0.039 | 55 |
| H | 0.078 | 0.039 | 0.019 | 18 |
| I | 0.039 | 0.019 | 0.009 | 1 |
| J | 0.019 | 0.009 | 0.0045 | 2 |
| K | 0.009 | 0.0045 | 0.0023 | 1 |

Experiment 2

### Time Course of Anti-HIV effect of CTC96

Using similar conditions as described above, virus was incubated with 0.625 mg/ml CTC96 for the indicated time period before 1,000-fold dilution and addition to cells.

Virus was incubated with four different concentrations of CTC96 for the indicated time periods, and then the extent of inactivation tested as previously described. Numbers in parentheses indicate results obtained in the initial analysis for the lowest dose (0.625 μg/ml) of CTC96.

TABLE 6
Neutralization of HIV by incubation with CTC 96

| Contact time | % Neutralization for [CTC96] - | | | |
|---|---|---|---|---|
| (minutes) | 5 μg/ml | 2.5 μ/mL | 1.25 μg/ml | 0.625 μg/ml |
| 64 | 100 | 100 | 94 | 80 (100) |
| 32 | 98 | 86 | 78 | 65 (76) |
| 16 | 78 | 72 | 61 | 56 (64) |
| 8 | 65 | 47 | 42 | 41 (56) |
| 4 | 52 | 25 | 17 | 25 (43) |
| 2 | 37 | 21 | 4 | 15 (7) |

### EXAMPLE 7

### Prophylactic Activity of CTC 96 Against Human Papillomavirus Type 11

The activity of CTC 96 on human papillomavirus type 11 (HPV-11) prior to infection was tested by using a subcutaneous human xenograft infection model in the severe combined immunodeficiency (SCID) mouse.

Methods

A. Protocol

Three different concentrations, 1%, 0.2% and 0.05%, of CTC 96 and a control (normal saline) were evaluated in HPV-11-infected internal human xenograft in the SCID mouse. The viral suspension was exposed for one hour with one of the four CTC 96 concentrations before being used to infect the human foreskin grafts.

Each experiment was done in quadruplicate. Twelve mice were used in each replicate. For each replicate, the four treatment groups were made of 3 mice (3 animals per cage). Each mouse received under the skin of each flank one HPV-11 infected foreskin fragment. Two foreskin donors were used, one for the fragments implanted under the left flank and a different one for the fragments implanted under the right flank. The HPV-11-infected grafts were left to grow for 12 weeks, while the mice weights were monitored every other week. At the end of the 12 weeks, the animals were sacrificed and the grafts recovered, measured and processed.

B. Experimental Design

Six to 7 week old male scid/scid mice were used

TABLE 7
Experimental Design

| | Replicate # | | | |
|---|---|---|---|---|
| Treatment | 1 | 2 | 3 | 4 |
| CTC 96 1% (Group 1) | 3 mice | 3 | 3 | 3 |
| CTC 96 0.2% (Group 2) | 3 | 3 | 3 | 3 |
| CTC 96 0.05% (Group 3) | 3 | 3 | 3 | 3 |
| CTC 96 0% (saline, control) (Group 4) - | 3 | 3 | 3 | 3 |

C. Drug Treatment

CTC 96 was applied to the virus directly, prior to infection. Just prior to exposure of the virus to CTC 96, 750 μl of normal saline was added to a vial containing 15 mg of dry CTC 96. The resulting solution contained 2% of CTC 96. This solution was diluted as indicated in Table 8, so that once mixed with the virus, the final concentration was 0%, 0.05%, 0.2%, or 1%.

CTC 96 was incubated for 1 hour at 37° C. with the viral suspension, which was then used to infect the human foreskin fragments.

TABLE 8
Preparation of the Viral Suspensions

| | Dilution 1 | Dilution 2 | Dilution 3 | Dilution 4 |
|---|---|---|---|---|
| Solute | 15 mg of | 150 μl of | 30 μl of | — |
| | dry CTC 96 | dilution 1 | dilution 1 | |
| Normal saline | 750 μl | 600 μl | 570 μl | 700 μl |
| Volume of the aliquot | 500 μl | 500 μl | 500 μl | 500 μl |
| of the corresponding | | | | |
| dilution | | | | |
| HPV-11 viral | 500 μl | 500 μl | 500 μl | 500 μl |
| suspension (lysate | | | | |
| 4/90; 1:19 dilution) | | | | |
| Final CTC 96 | 1% | 0.2% | 0.05% | 0% |
| concentration (w/v) | | | | |
| Treatment Group | 1 | 2 | 3 | 4 |

D. Grafting

Neonatal foreskins from routine circumcision were collected at the nursery of a local hospital and placed in transport medium (Minimum Essential Medium, penicillin 25,000 U/ml, streptomycin 25 mg/mL), In a Petri dish, the foreskin's occluded side was removed using a scalpel, leaving the exposed skin prepared as a split-thickness graft. The foreskin was punched out using a 3 mm biopsy punch. One fragment was fixed in buffered formalin to serve as control in the histologic and immunocytochemical analyses. 34 fragments were snap frozen in liquid nitrogen, to serve as controls in the RT-PCR assay. The other fragments were split equally into four groups. Each group of fragments was dispensed in one of the vials containing 500 μl of pretreated viral suspension.

Two foreskin donors collected within 24 hours of the procedure, were used in each replicate experiment. Each viral suspension received the foreskin fragments of both foreskin donors. To recognize the foreskin donors, in each experiment we used a white baby and a black baby. The fragments were vortexed in the suspension and left to incubate for 60 minutes at 37° C. before being grafted.

For the grafting, the mouse was anesthetized in an anesthesia chamber with 5% halothane to 2 liters of oxygen, until no corneal or pedal reflexes could be elicited. The animal was removed from the chamber, and the halothane adjusted to 2-2.5% to 1 liter of oxygen for continued administration via nose cone for the duration of the surgical procedure. The animals ears were notched for identification. The back skin was prepped with a povidone iodine swab. A 1 cm vertical incision of the skin was made on the flank with scissors. Through that incision, the subcutis was bluntly dissected with closed scissors caudad to the wound. One foreskin fragment from the white donor was inserted in the caudad pocket, epidermis facing the mouse's skin, mucosal side in contact with the musculo-fascial plane (panniculus camosus) of the mouse. The incision was closed with two metal clips. The other side was grafted in the same manner with one foreskin fragment from the black donor.

E. Monitoring, Euthanasia and Graft Collection

The animals were monitored weekly. They were weighed at the time of grafting, and every other week during the 12 weeks of the experiment.

Animals were to be prematurely euthanitized if during monitoring any of the following criteria were met:

greater than 10% weight loss;

bleeding or bruising;

inability to maintain righting reflex;

inability to move about;

inability to eat or drink;

tumor greater than 30% of the animal's body weight.

The animals were otherwise sacrificed by cervical dislocation 12 weeks after graft implantation. Length, width, and height of the graft were measured and recorded. The graft were then removed and split in at least two parts. One part was fixed in buffered formalin. The other part was either left as is, or subdivided for subsequent analyses in as many fragments as size permitted. These fragments were placed in a sterile labelled vial and frozen in liquid nitrogen before being transferred to a −80° C. freezer.

F. Premature Deaths

Those animals dying unexpectedly before the date of the scheduled euthanasia had their grafts measured and fixed in formalin, if body decomposition was not too advanced.

G. Statistical Methods

1. Data Management

Patient, animal, and experimental data were entered in a database maintained on a personal computer. Data handling and analysis was done primarily with the statistical software STATISTICA/5.1w (Statsoft Inc.), StatXact (Cytel Corp. Cambridge, Mass.) software was used for the statistical analysis by exact methods. The Minitab software (Minitab Inc.) was used for the randomization. The statistical power calculations were carried out with the software PASS 6.0 for Windows (NCSS, Kaysville, Utah).

2. Design and Analysis of the Primary Endpoint (Graft Size)

We conducted the Efficacy studies using a factorial design. The two factors were Treatment (fixed factor) and, as a concomitant variable, the Replicate factor to take into account the well-established variability introduced by the foreskin donor. The Replicate factor was treated as a fixed factor since it is used for adjustment purposes in the specific experiment. Therefore, the analysis was that of a 2-way crossed, between-subjects ANOVA model. There were 3 replications per cell, corresponding to the 3 animals in a cage.

The cages were sequentially numbered upon arrival in the xenograft facility. Treatment was assigned to the cages from a list of random numbers generated for each replicate and matched to the case sequence number.

For each mouse, at the end of the experiment, we calculated a composite geometric mean diameter (cGMD) of the grafts, which was chosen before the experiment as our primary endpoint:

cGMD=(3√(length×weidth×height)_{left side}+³√(length×width×height)_{right side})/2

In case one of the two grafts was missing, the denominator was one and if both grafts were missing, then a cCMD was not calculated for that particular animal.

Pairwise comparisons between the CTC 96-treated groups and the vehicle-treated group was done using the Tukey Honest Significant Difference test, modified by Spjotvoll & Stoline for unequal n's. We also conducted an analysis for linear or quadratic trend across the different concentrations of CTC 96.

For α=0.05 and we had calculated a statistical power of 0.80 to detect an effect size of 0.59. The effect size being defined as the difference between the largest and smallest mean cGMDs divided by the within-cell standard deviation.

In all statistical analyses two-sided p values equal or less than 0.05 were considered significant.

Results

A. Animals

Forty-eight animals were grafted according to protocol. Seven animals died or had to be sacrificed before the end of the experiment. The monitored the mortality of the animals in the different groups was monitored and did not detect a significant imbalance that would have led to the premature euthanasia of the animals. This mouse mortality rate is not excessive. SCID mice are fragile, and a major cause of mortality is the spontaneous development in 15% of animals aged 4 to 17 months of thymic lymphomas that readily metastasize.

B. Grafts

82 grafts were implanted in a the 41 mice that survived until the end of the experiment and 77 (94%) were present at euthanasia. Table 9 summarizes the distribution of the grafts present at euthanasia according to treatment groups. Three mice had only one surviving graft. Unfortunately, the only mouse with no surviving grafts belonged to the CTC 96 0% group of the second replicate, a group that had already lost two animals from premature deaths. Consequently, there was no control group for the second experiment. Therefore the efficacy analysis of CTC 96 that is presented is based on replicates 1, 3 and 4. All animals (4 replicates) are included in the Toxicity Analysis.

TABLE 9
Grafts Present at Euthanasia

| | | Number of | | | |
|---|---|---|---|---|---|
| | | surviving | | | |
| | | mice | | | |
| | | with the | | | |
| | | following | | | |
| | | number of | | | |
| | Treatment | grafts | | | Row |
| Replicate | Group | 0 | 1 | 2 | Total |
| 1 | CTC 96 0% | 0 | 0 | 3 | 3 |
| | CTC 96 0.05% | 0 | 0 | 3 | 3 |
| | CTC 96 0.2% | 0 | 0 | 2 | 2 |
| | CTC 96 1% | 0 | 0 | 3 | 2 |
| | Total | 0 | 0 | 11 | 11 |
| 2 | CTC 96 0% | 1 | 0 | 0 | 1 |
| | CTC 96 0.05% | 0 | 1 | 2 | 3 |
| | CTC 96 0.2% | 0 | 0 | 2 | 2 |
| | CTC 96 1% | 0 | 0 | 3 | 3 |
| | Total | 1 | 1 | 7 | 9 |
| 3 | CTC 96 0% | 0 | 1 | 2 | 3 |
| | CTC 96 0.05% | 0 | 0 | 3 | 3 |
| | CTC 96 0.2% | 0 | 0 | 2 | 2 |
| | CTC 96 1% | 0 | 0 | 3 | 3 |
| | Total | 0 | 1 | 10 | 11 |
| 4 | CTC 96 0% | 0 | 0 | 3 | 3 |
| | CTC 96 0.05% | 0 | 0 | 2 | 2 |
| | CTC 96 0.2% | 0 | 0 | 2 | 2 |
| | CTC 96 1% | 0 | 1 | 2 | 3 |
| | Total | 0 | 1 | 9 | 10 |

| | | | | |
|---|---|---|---|---|
| Column Total | 1 | 3 | 37 | 41 |

C. Effect of CTC 96 on Graft Size

The composite Geometric Mean Diameter of the grafts was our primary endpoint in this evaluation of CTC 96. Table 10 provides the summary statistics of this measurement. The highest mean (or median) cGMD was in the group where the infecting virus was treated with saline only (CTC 96 0% group). FIG. 1 displays the cGMDs of the four treatment groups.

TABLE 10
Summary Statistics on the Composite Geometric Mean Diameters
(cGMD) of the Grafts (mm)

| Treatment | | | Standard | Lower | | Upper |
|---|---|---|---|---|---|---|
| Groups | Means | N | Deviations | Quartile | Median | Quartile |
| CTC 96 0% | 2.58 | 9 | .808 | 2.22 | 2.89 | 2.96 |
| CTC 96 0.05% | 2.02 | 8 | .179 | 1.94 | 2.01 | 2.13 |
| CTC 96 0.2% | 1.86 | 6 | .196 | 1.70 | 1.88 | 2.05 |
| CTC 96 1% | 1.95 | 9 | .139 | 1.82 | 1.94 | 2.08 |
| All | 2.13 | 32 | .52 | 1.83 | 2.07 | 2.22 |

Table 11 shows the statistical analysis by ANOVA of these results. The graft size varied significantly according to the treatment applied to the viral inoculum (p=0.004). Furthermore, there was a dose-response effect (p=0.002). A pairwise comparison of the cGMDs in the treatment groups (Table 12) establishes that regardless of the CTC 96 concentration there was a significant effect on the infectivity of HPV-11 when compared to the control (no CTC 96).

TABLE 11
ANOVA of the Effects on Graft Composite
Geometric Diameter (cGMD)

| | df | MS | df | MS | | |
|---|---|---|---|---|---|---|
| Effect | Effect | Effect | Error | Error | Fn | p level |
| Treatment* | 3 | 0.8690 | 20 | 0.1438 | 6.0467 | 0.0043 |
| Replicate | 2 | 0.4941 | 20 | 0.1438 | 3.437 | 0.052 |
| Treatment × | 6 | 0.2888 | 20 | 0.1438 | 2.009 | 0.11 |
| Replicate | | | | | | |
| Interaction | | | | | | |

*There was a dose-response effect (p = 0.0023; by test for linear trend)

CTC 96 on graft size. Although the analysis of the experiment was compromised by the inability to use the results of one of the four replicate experiments, the results were clear: CTC 96 regardless of the concentration used, limited the HPV-11-inducted growth of the grafts. Even the 0.05% concentration was effective. It is impossible to determine at this stage of the analysis the concentration below which CTC 96 loses its effect on graft size. This question might be answered using other endpoints, histology, capsid protein expression (immunocytochemistry), and viral transcription (RT-PER). This analysis is in progress.

CTC 96 has no effect on the animal's mortality or weight gain. This was expected since the drug was not directly administered to the animals. It was used only to inactivate HPV-11 in vitro.

I claim:

1. A method for prophylactically reducing the risk of transmission of a specific virus to a recipient and protecting the recipient from infection by the specific virus comprising topically applying to an appropriate site on the recipient a specific virus prophylactic effective amount of a compound having the structure 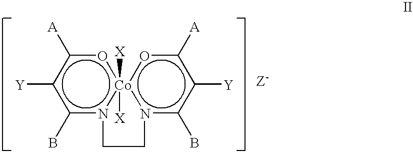
wherein each
A may be the same or different and is an alkyl group, a phenyl group or a substituted derivative of a phenyl group;
Y may be the same or different and is hydrogen, an unbranched alkyl group, a halide or a group having the structure
wherein R is hydrogen, an alkoxide group, an alkyl group, or OH;
B may be the same or different and each is hydrogen or an alkyl group;
Z⁻ is a soluble, pharmaceutically acceptable negative ion, and
X may be the same or different and is an axial ligand selected from the group consisting of moieties having the formula: 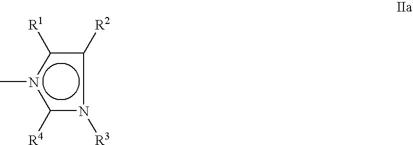
wherein R¹, R², R³, and R⁴ may be the same or different and may be hydrogen or lower alkyl having from 1 to 4 carbon atoms;
with the proviso that R¹, R², R³, and R⁴ are of a sufficiently small size so as not to prohibit the attachment of the axial ligand to the Co atom due to steric hindrance.

2. The method of claim 1 wherein the specific virus is selected from the group consisting of adenovirus, human immunodeficiency virus (HIV), human papillomavirus (HPV) and varicella-zoster virus.

3. The method of claim 1 wherein the appropriate site is that site on the recipient which is exposed to the specific virus.

4. The method of claim 1 wherein the compound is applied to a mucus membrane of the recipient.

5. The method of claim 1 wherein the compound is applied to the eye.

6. The method of claim 1 wherein the compound is applied to the respiratory tract of the recipient.

7. The method of claim 1 wherein the compound is applied from 8 hours before to about 6 hours after exposure to the specific virus.

8. The method of claim 1 wherein the compound is applied from about 1 hour before to about 6 hours after exposure to the specific virus.

9. The method of claim 1 wherein the compound is applied from about 5 minutes before to about 5 minutes after exposure to the specific virus.

10. The method of claim 1 wherein the virus is a specific adenovirus selected from the group consisting of Ad1, Ad2, Ad3, Ad4, Ad5, Ad6, Ad7, Ad8, Ad11, Ad14, Ad19, Ad21, Ad34, Ad35, Ad37, Ad40 and Ad41.

11. The method of claim 1 wherein the compound is Compound 96.

12. The method of claim 1 wherein the step of topically applying the compound is performed by contacting the recipient with an aerosol of the compound.

13. A method for disinfecting a liquid containing a specific virus comprising adding to the liquid a specific virus prophylactic effective amount of a compound having the structure 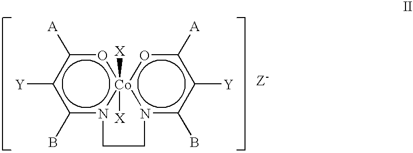
wherein each
A may be the same or different and is an alkyl group, a phenyl group or a substituted derivative of a phenyl group;
Y may be the same or different and is hydrogen, an unbranched alkyl group, a halide or a group having the structure
wherein R is hydrogen, an alkoxide group, an alkyl group, or OH;
B may be the same or different and each is hydrogen or an alkyl group;
Z⁻ is a soluble, pharmaceutically acceptable negative ion, and
X may be the same or different and is an axial ligand selected from the group consisting of moieties having the formula: 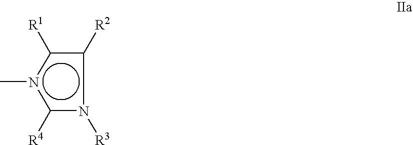
wherein R¹, R², R³, and R⁴ may be the same or different and may be hydrogen or lower alkyl having from 1 to 4 carbon atoms;
with the proviso that R¹, R², R³, and R⁴ are of a sufficiently small size so as not to prohibit the attachment of the axial ligand to the Co atom due to steric hindrance.

14. The method of claim 13 wherein the specific virus is selected from the group consisting of adenovirus, human immunodeficiency virus (HIV), human papillomavirus (HPV) and varicella-zoster virus.

15. The method of claim 13 wherein the compound is added in an amount of about 0.00005 to about 5% by weight of the liquid.

16. The method of claim 13 wherein the compound is added in an amount of about 0.005 to about 5% by weight of the liquid.

17. The method of claim 13 wherein the compound is added in an amount of about 0.005 to about 2% by weight of the liquid.

18. The method of claim 13 wherein the compound is added in an amount of about 0.01 to about 2% by weight of the liquid.

19. The method of claim 13 wherein the liquid is a growth media or a blood-derived product.

20. The method of claim 2 wherein the virus is a specific Human Immunodeficiency Virus selected from the group consisting of HIV-1 and HIV-2.

21. The method of claim 2 wherein the specific virus is selected from the group consisting of HPV-1, HPV-2, HPV-3, HPV-4, HPV-6, HPV-7, HPV-10, HPV-11, HPV-16, HPV-18, HPV-31 and HPV-45.
